SEQUENCE LISTING (1 sequence)

SEQ ID NO: 1 - protein, 5 aa, Artificial Sequence
  synthetic
GLDGA

(19) United States
(12) Patent Application Publication
(10) Pub. No.: US 2022/0339097 A1
(43) Pub. Date: Oct. 27, 2022

(54) DISCONTINUOUS GUMMY DELIVERY SYSTEMS

(71) Applicant: GELITA AG, Eberbach (DE)

(72) Inventors: Mindi McKibbin, Olathe, KS (US); Michelle A. Montgomery, Jefferson, SD (US); Ginger Waller, Hornick, IA (US); Eric Yezdimer, Sioux City, IA (US)

(21) Appl. No.: 17/242,030

(22) Filed: Apr. 27, 2021

(51) Int. Cl. A61K 9/00; A61K 47/42; A61K 9/10; A61K 47/26; A61K 47/10; A61K 47/12; A61K 31/07; A61K 31/51; A61K 31/525; A61K 31/4415; A61K 31/714; A61K 31/375; A61K 31/593; A61K 31/355; A61K 31/122; A61K 31/4188; A61K 31/14; A61K 33/18; A61K 31/198; A61K 35/60; A61K 36/48; A23G 3/44; A23G 3/42; A23G 3/36; A23G 3/40; A23G 3/48; A23G 3/34

(52) U.S. Cl. CPC A61K 9/0056; A61K 47/42; A61K 9/10; A61K 47/26; A61K 47/10; A61K 47/12; A61K 31/07; A61K 31/51; A61K 31/525; A61K 31/4415; A61K 31/714; A61K 31/375; A61K 31/593; A61K 31/355; A61K 31/122; A61K 31/4188; A61K 31/14; A61K 33/18; A61K 31/198; A61K 35/60; A61K 36/48; A23G 3/44; A23G 3/42; A23G 3/368; A23G 3/362; A23G 3/40; A23G 3/48; A23G 3/0019; A23V 2002/00

(57) ABSTRACT

Disclosed is an edible, discontinuous gummy delivery system comprising collagen peptide particles with an average particle size from 5 to 500 μm and an average molecular weight from 1,000 to 10,000 Da dispersed in an elastic matrix, wherein the collagen peptide particles are undissolved or no more than partially dissolved in the elastic matrix, and wherein the collagen peptide particles are present in an amount from 8 to 45% of a total mass of the delivery system. Methods of preparing the gummy delivery systems are also disclosed.

Incorporated by reference in its entirety herein is a computer-readable nucleotide/amino acid sequence listing submitted concurrently herewith and identified as follows: One 327 Byte ASCII (Text) file named “751001_ST25.txt,” dated Apr. 26, 2021.

### BACKGROUND OF THE INVENTION

Gummy delivery systems may be useful for a variety of applications including, for example, as a carrier for edible nutritional supplements. Despite advancements in the field, many conventional gummy formulations have not successfully provided a product that is both acceptable to consumers and cost-efficient to prepare. Accordingly, there is a need for improved gummy delivery systems.

### BRIEF SUMMARY OF THE INVENTION

An aspect of the invention provides an edible, discontinuous gummy delivery system comprising collagen peptide particles with an average particle size from 5 to 500 μm and an average molecular weight (MW) from 1,000 to 10,000 Da dispersed in an elastic matrix, wherein the collagen peptide particles are undissolved or no more than partially dissolved in the elastic matrix, and wherein the collagen peptide particles are present in an amount from 8 to 45% of a total mass of the delivery system.

Still another aspect of the invention provides a method of preparing any of the inventive edible, discontinuous gummy delivery systems described herein, the method comprising: dispersing collagen peptide particles in an edible liquid dispersion medium to produce a dispersion; combining the dispersion with a sweetener solution and an aqueous gelation solution to produce a formulation liquor; and molding and drying the formulation liquor into a chewable shape, thereby producing the edible, discontinuous gummy delivery system.

### BRIEF DESCRIPTION OF THE SEVERAL VIEWS OF THE DRAWINGS

FIG. 1 presents a cross-sectional image of a discontinuous gummy delivery system according to an aspect of the invention taken under 60× magnification which illustrates the discontinuous microstructure. The scale bar on the bottom right side of FIG. 1 denotes a 100 μm length.

FIG. 2 is a graph illustrating the hardness (g) over time (weeks) of the discontinuous gummy delivery system according to an aspect of the invention (Formulations I-IV) as compared to conventional gummy formulations (Control I and II).

FIG. 3 is a graph showing the luminosity values of gummy delivery systems according to aspects of the invention (Formulations SF1-SF4) and controls (SF-STD1 and SF-STD2).

FIG. 4 is a graph showing the luminosity values of gummy delivery systems according to aspects of the invention (Formulations SC1-SC4) and controls (SC-STD1 and SC-STD2).

### DETAILED DESCRIPTION OF THE INVENTION

Aspects of the invention provide an edible, discontinuous gummy delivery system comprising collagen peptides and methods for producing the same. The inventive gummy delivery systems comprise a unique discontinuous microstructure that may reduce undesirable chemical interactions within the delivery system and may reduce or eliminate the need for excess water to dissolve the collagen peptides. The inventive gummy delivery systems may provide any one or more of a variety of advantages including, for example, a capacity for delivering a higher load of collagen peptides per serving, more resistance to hardening as compared to conventional gummy formulations, and a taste and texture that is acceptable to the palate and which is noticeably less bitter. The inventive gummy delivery systems may also have a significantly lower water activity than conventional gummy formulations, which may improve its resistance to microbial contamination. The inventive gummy delivery systems may be produced by a multistep method that first disperses collagen peptides in an edible dispersion medium, which may then be combined with a separately prepared sweetener solution and aqueous gelation solution, molded into suitable chewable shapes, and dried.

The inventive gummy delivery systems may ameliorate one or more of the disadvantages of conventional gummy formulations described in more detail below. Numerous attempts have been made to formulate acceptable gummy delivery systems that contain active ingredients. For example, U.S. Pat. Nos. 4,778,676; 4,882,152; and 4,882,154 describe a chewable composition providing a smooth texture devoid of graininess for delivering active ingredients. U.S. Pat. No. 5,928,664 describes a consumable gummy delivery system where an elastic, continuous glycerinated gelatin matrix is prepared by evaporating water through an increased temperature step (85-100° C.) and then combining the matrix with active ingredient(s) contained within a saccharide-based or water-soluble cellulose shearform matrix. Key in these applications is the requirement to remove significant fractions of the moisture in the formulation during a drying step. WO 2010/151275 describes a chewable composition for delivering pharmaceutical ingredients or supplements that are prepared by directly mixing active ingredients with a gelatin solution.

Many of these conventional gummy formulations, however, have not been wholly successful in providing both an acceptable product for consumers and a cost-efficient method of production. The scope of specific chemistries comprising known pharmaceutical and nutraceutical active compounds is incredibly diverse and, as a consequence, many unanticipated limitations may be encountered because of complex interactions between the active ingredients and those comprising the delivery system.

Collagen peptides (also referred to as “collagen hydrolysate” or “gelatin hydrolysate”) are active ingredients with proven health benefits (Postlethwaite et al., PNAS, 75: 871-875 (1978); Oesser et al., J. Nutrit., 129: 1891-1895 (1999); Adam, Therapiewoche, 41: 2456-2461 (1991); Benito-Ruiz et al., Int. J. Food Sci. Nutrit., 60: 99-113 (2009); and Moskowitz, Sem. Arthrit. Rheum., 30: 87-99 (2000)). Collagen peptides are derived from collagen present in an animal source material, usually of a porcine, fish, or bovine origin. Collagen peptides may be produced by hydrolyzing the collagen, either thermally or through enzymatic means, to such a degree that the material loses the ability to form a gel when dissolved in an aqueous solution. Collagen peptides are distinct from gelatin, which forms gels at various concentrations and temperatures. The average molecular weights of collagen peptides are typically ≤15 kDa. Collagen peptides are highly water soluble and typically dissolve in cold water readily. The water solubility of collagen peptides can exceed 40% by weight. Collagen peptides may be commercially available as dried powders and may be marketed under any of a variety of trademarked names including, but not limited to, VERSIOL®, TENDOFORTE®, FORTIGEL®, PEPTIPLUS®, BODYBALANCE®, PETAGILE® and FORTIBONE® (all available from Gelita AG, Eberbach, Germany).

There are several challenges to the development of gummy delivery systems with collagen peptides that have not been satisfactorily addressed by conventional gummy formulations. For example, the recommended daily doses of the collagen peptide products VERISOL® and FORTIGEL® are 2,500 mg and 5,000 mg, respectively. These doses may be difficult to achieve in an acceptable number of individual gummy servings because the average mass of a standard gummy is typically from 2,000 to 3,000 mg. To achieve the higher daily dose of collagen peptides in a more practical number of individual gummy units, it may be desirable to utilize formulations with a higher load of collagen peptides (>20% collagen peptide by weight).

Moreover, collagen peptides can exhibit a bitter taste. Conventional gummy formulations containing higher doses of collagen peptides (>700 mg per serving) can have an undesirable bitter taste that may require special flavoring considerations. Conventional gummy formulations using high doses of dissolved collagen peptides may also harden during storage, causing an unappealing texture and becoming difficult to chew. The gummy texture is typically measured in terms of hardness (reported in grams). Hardness values of under 150 g are considered highly desirable to customers. Textures from 150 to 300 g are considered acceptable. Textures over 300 g are considered undesirable or unacceptable. A conventional sugar-containing gummy candy, without collagen peptides, typically may see an increase in hardness from 100 g to 300 g over 6 months of storage. Conventional gummy formulations containing a high load of dissolved collagen peptides (>20% collagen peptide) harden much faster during storage and can exceed 425 g after four months of storage.

The hardness and shelf-life of a gummy delivery system may be influenced by its water activity. Water activity is a measurement of unbound water and is defined as the ratio of the water vapor pressure from a material to the water saturation vapor pressure at the same temperature. Because unbound water molecules are available to act as a solvent and are free to participate in chemical or microbiological reactions, it has been established by the food industry as a good predictor of product lifetime and spoilage rates. Higher water activities may also be indicative of higher rates of water loss (i.e., the drying out of a product) and the hardening of materials where water acts as a plasticizer. Most confectionary products are desired to have a water activity of from 0.50 to 0.75 units. Microbial activity may be largely reduced at water activities under 0.60 units. Gummy products with water activities under 0.60 units are, therefore, highly preferred as the product is expected to have good longevity during extended storage. Conversely, water activities higher than 0.75 units are expected to perform worse during storage, with microbial growth becoming a major concern at water activities greater than 0.85 units.

From a manufacturing perspective, the amount of water necessary is a consideration in a gummy formulation for the delivery of collagen peptides. Gummy formulations are usually produced by the preparation of a sweetener solution, comprising sugar or a sugar-substitute at high concentrations (≥80 Brix) and a gelation solution utilizing high Bloom gelatin (˜250 g Bloom) at concentrations of around 30-40%. The sweetener solution is typically boiled at high temperatures (>100° C.) to remove excess water. The gelation solution may be prepared by dissolving a gelatin powder or dried granular gelatin in water at a temperature from 60 to 70° C. Gelatin degrades at temperatures in excess of 60° C., so the use of two separate solutions is commonplace in the industry to maintain the gelation strength of the gelatin. The sweetener and gelatin solutions are then combined, followed by the addition of a tailoring cocktail comprising components for one or more of pH, flavor, and color adjustments. The precise order of mixing can be varied as practical, provided the temperature of the solutions are compatible with the chemical stability of the components being added. One or more of nutritional, nutraceutical, and pharmaceutical active ingredients can also be incorporated into the sweetener solution, gelation solution, or tailoring cocktail as limited by the chemical stabilities of those ingredients. In the case of collagen peptides, it is common practice to fully dissolve the collagen peptides into the gelation solution, prior to combination with the sweetener solution or tailoring cocktail.

Collagen peptides, being derived from collagen, have a similar sidechain chemistry as compared to their parent gelatin molecules. The lower MW of the collagen peptides, however, results in a higher occurrence of zwitterionic charges per amino acid in a peptide chain, in addition to significantly less chain entanglements. Collagen peptides, therefore, are usually the dominant competitor for water in aqueous gelatin solutions. For high load collagen peptide-containing gummy formulations, significantly more water may be needed to both fully dissolve both the gelatin and the collagen peptides during the preparation of the gelatin solution. In conventional gummy formulations, gelatin is responsible for the gel formulation of the gummy. Failure to fully dissolve the gelatin may result in poorly gelled products or a failure of the formulation liquor to properly set when molded. The required higher water content may translate into higher water activities, as more water may be present in the gel matrix. Additional or extended drying processes, to remove the excess water, may also be required if the water activity of a gummy formulation is too high. In cases where excess water must be removed, longer processing times and lower production throughput rates may be needed and may be economically unfavorable.

Glycerin also competes significantly for available water, thus homogenous aqueous mixtures of glycerin, collagen peptides, and gelatin also may require a significantly higher water content to produce workable gummy formulation liquors. To illustrate this, Table 1 below summarizes a series of experiments in which different ratios of collagen peptides, glycerin, gelatin, and water were combined at 70° C. for 4.5 hours in an attempt to prepare a homogenous and continuous solution of a conventional gummy formulation. While in conventional gummy formulations, a ratio of 2 parts water to 1 part dried gelatin is normally used to prepare the gelatin solution, this is not possible in the presence of collagen peptides and glycerin. Ratios of 10 parts water to 1 part dried gelatin may be required to obtain a homogenous and continuous gelation solution that is capable of setting into an acceptable gel. This is because both the dissolved collagen peptides and glycerin compete for the available water, reducing the amount of water needed to fully dissolve the gelatin. Gelation strength is fundamentally derived from the entanglements of the gelatin molecular chains. The formation of entanglements may be significantly hindered if enough water is not available to fully dissolve the dried gelatin starting material and produce a homogenous and continuous solution. Conventional gummy formulations for delivering active ingredients using a continuous and glycerated gelatin matrix, when collagen peptides serve as the active ingredient, is therefore limited and represents a current challenge to the industry.

TABLE 1
Table 1: A high moisture content may be required to
produce homogenous and settable gelatin mixtures

| | Example Solutions | | | | | | | |
|---|---|---|---|---|---|---|---|---|
| | T1 | T2 | T3 | T4 | T5 | T6 | T7 | T8 |
| Collagen | 34.7% | 27.8% | 25.4% | 23.5% | 22.4% | 21.9% | 21.5% | 21.1% |
| peptides | | | | | | | | |
| (VERISOL ®) | | | | | | | | |
| Glycerin | 43.3% | 34.7% | 31.7% | 29.4% | 27.9% | 27.4% | 26.9% | 26.4% |
| Gelatin | 7.4% | 5.9% | 5.4% | 5.0% | 4.8% | 4.7% | 4.6% | 4.5% |
| Water | 14.6% | 31.5% | 37.4% | 42.0% | 44.9% | 46.0% | 47.0% | 48.0% |
| Phase | Yes | Yes | Yes | Yes | Yes | Yes | Yes | Yes |
| separated | | | | | | | | |
| Settable | Failed | Failed | Failed | Failed | Failed | Failed | Failed | Passed |

Significant discoloration can also occur during storage of collagen peptide-containing gummy formulations, resulting in brown or black products that appear spoiled or unsafe to consume. This discoloration is caused by the Maillard browning reaction that occurs between the free amines of the peptide chains and sugars present in the gummy matrix. In the case of sugar-containing gummy delivery systems comprising high concentrations of collagen peptides, this effect can be quite pronounced with noticeable discolorations occurring within weeks. Sugar-free formulations can reduce the prevalence of this reaction in high-load collagen peptide gummy formulations, but does not provide a complete solution as negative gastrointestinal issues can occur with the overuse of sugar-substitutes, and some consumers prefer the taste of sugar containing gummy formulations.

Collagen peptides also chemically react with gelatin through the formation covalent bonds between their polymer chains, otherwise referred to as “cross-linking.” Cross-linking may reduce the biological efficacy of the collagen peptides by significantly increasing their MW, decreasing their solubility and negatively affecting their pharmacokinetic profile.

The inventive gummy delivery systems may ameliorate one or more of the disadvantages of conventional gummy formulations described above. Aspects of the invention provide a consumable (e.g., edible), gummy delivery system that comprises collagen peptides contained within a unique discontinuous microstructure. The inventive gummy delivery system may be an elastic, opaque matrix that can dissolve in one or both of the mouth and stomach. The discontinuous microstructure may comprise undissolved or no more than partially dissolved collagen peptide particles. Because these particles of collagen peptides are undissolved or not fully dissolved, the amount of water necessary to produce workable formulation liquors may be reduced. Surprisingly, the graininess of the discontinuous microstructure may only be slightly noticeable, if at all, when chewed. Experimentation has also found that the inventive gummy delivery systems may provide a more acceptable palate via a softer texture as compared to conventional gummy delivery systems that utilize similar concentrations of fully dissolved collagen peptides. The taste of the inventive gummy delivery systems may also be noticeably less bitter than conventional gummy formulations that have the same chemical composition (by weight), but are transparent and lack the discontinuous microstructure of the inventive gummy delivery systems.

The discontinuous microstructure may also reduce the contact between the collagen peptides and the surrounding elastic matrix. The rate of Maillard browning can be reduced because of reduced direct molecular contact between the undissolved or no more than partially dissolved collagen peptides and the sugars present in the sweetener solution. The molecular contact between the collagen peptides and gelatin molecules may also be reduced, which may reduce the rate of undesirable cross-linking reactions.

The lower amount of water present in the inventive gummy delivery systems may also reduce the water activity. The lower water activity of the inventive gummy delivery systems may result in significantly less water loss during storage and may maintain the desired soft texture over the shelf life of the inventive gummy delivery systems. Formulations utilizing fully dissolved collagen peptides may require more water to obtain workable solutions and thus may have higher water activities unless additional costly steps are taken to remove the excess water.

An aspect of the invention provides an edible, discontinuous gummy delivery system comprising collagen peptide particles dispersed in an elastic matrix. The collagen peptide particles are undissolved or no more than partially dissolved in the elastic matrix.

The collagen peptide particles may have an average particle size from 5 μm to 500 μm. In an aspect of the invention, the collagen peptide particles may have an average particle size of 50 μM to 450 μm, 100 μm to 400 μm, or 150 μm to 350 μm. For example, the collagen peptide particles may have an average particle size of 5 μm, 50 μM, 100 μm, 150 μm, 200 μm, 250 μm, 300 μm, 350 μm, 400 μm, 450 μm, or 500 μm, or a range defined by any two of the foregoing values.

The collagen peptide particles may have an average MW from 1,000 Da to 10,000 Da. In aspects of the invention, the collagen peptide particles may have an average MW from 2,000 Da to 7,000 Da or 3,000 Da to 6,000 Da. For example, the collagen peptide particles may have an average MW of 1,000 Da, 1,500 Da, 2,000 Da, 2,500 Da, 3,000 Da, 3,500 Da, 4,000 Da, 4,500 Da, 5,000 Da, 5,500 Da, 6,000 Da, 6,500 Da, 7,000 Da, 7,500 Da, 8,000 Da, 8,500 Da, 9,000 Da, 9,500 Da, 10,000 Da or a range defined by any two of the foregoing values. Preferably, the collagen peptide particles have an average MW from 1,200 Da to 6,000 Da. The average MW values disclosed herein refer to the weight-average molecular weight. This can be determined, for example, by size exclusion chromatography and a set of standard calibration peptides. In one aspect, the size exclusion chromatography utilizes a silica-based TOSOH TSKgel G2000 SWxl column (Tosoh Bioscience LLC, King of Prussia, Pa.) (5 μm particle size, 7.8×300 mm), a mobile phase of 200 mM sodium monobasic phosphate buffer adjusted to pH=5.3 with NaOH, a flow rate of 0.5 mL/min and a detection wavelength of 214 nm. The set of calibration peptides can be defined as having molecular weights of 55 kDa, 38 kDa, 25 kDa, 19 kDa, 13.5 kDa, 3.5 kDa and 432 Da, wherein the 55 kDa-3.5 kDa peptides are produced by the selective cleavage of acid soluble, bovine hide collagen with cyanogen bromide and the 432 Da peptide is produced synthetically to have the amino acid sequence GLDGA (SEQ ID NO: 1) (zwitterionic).

The collagen peptide particles are present in an amount from 8 to 45% of a total mass of the delivery system. In aspects of the invention, the collagen peptide particles are present in an amount from 10 to 40% or 15 to 35% of a total mass of the delivery system. For example, the collagen peptide particles may be present in an amount of 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, or a range defined by any two of the foregoing values, of a total mass of the delivery system.

In an aspect of the invention, the elastic matrix of the gummy delivery system comprises one or more edible gelling agents, one or more sweeteners, and one or more edible hygroscopic agents.

The one or more gelling agents may be any of a variety of agents suitable for human or animal consumption that form a gel when dissolved in liquid. In an aspect of the invention, the edible gelling agent is a hydrocolloid. In an aspect of the invention, the one or more edible gelling agents are selected from the group consisting of gelatin, starch, pectin, agar, locust bean gum, gellan gum, gum arabic, carrageenan, alginate, modified cellulose, and xanthan gum. The gelling agent is not limited to these exemplary gelling agents. In a preferred aspect, the gelling agent is gelatin or starch.

The gelatin can be derived from any of a variety of sources including, but not limited to, porcine skin, bovine hide, bovine hide-spilt, bovine bone, and fish. The term “gelatin” refers to collagen that has been denatured from its native coiled microstructure and has the ability to form aqueous gels. The gelatin can be either type A (acid treated) or type B (base treated). The gelatin may have a Bloom value from 100 to 300 g. In a preferred aspect of the invention, the gelatin is derived from porcine skin. In a preferred aspect of the invention, the gelatin has a Bloom value of 240 to 260 g.

The one or more sweeteners may be any of a variety of substances suitable for human or animal consumption useful for sweetening the taste of food or drink. The sweetener can be, for example, a carbohydrate, sugar, or sugar substitute. Examples of sweeteners which may be useful for the inventive gummy delivery systems include, but are not limited to, maltitol syrup, xylitol, sugar, and corn syrup. Maltitol syrup and xylitol are useful sugar substitutes.

A hygroscopic agent is one that readily attracts water from its surroundings. Any of a variety of hygroscopic agents suitable for human or animal consumption may be useful for the inventive gummy delivery systems. In an aspect of the invention, the one or more edible hygroscopic agents is selected from the group consisting of glycerin, sorbitol, anhydrous glycerin, and anhydrous sorbitol. The hygroscopic agent is not limited to these exemplary hygroscopic agents.

In an aspect of the invention, the gummy delivery system further comprises one or more nutritional supplement(s), nutraceutical supplement(s), and/or pharmaceutical active ingredient(s). In an aspect of the invention, the gummy delivery system further comprises one or more nutritional supplements selected from the group consisting of vitamin A, vitamin B1, vitamin B2, vitamin B6, vitamin B12, vitamin C, vitamin D, vitamin E, vitamin K, biotin, choline, iodine, folic acid, histidine, isoleucine, leucine, lysine, methionine, phenylalanine, threonine, tryptophan, valine, hydroxyproline, calcium, zinc, iron, phosphorous, pea protein, soy, whey and fish oil.

In an aspect of the invention, the gummy delivery system further comprises one or more ingredients selected from the group consisting of flavoring ingredient(s), coloring ingredient(s), and pH adjusting ingredient(s). In a preferred aspect of the invention, the gummy delivery system comprises natural and/or artificial coloring ingredients to adjust the color and hue of the gummy delivery system. Such coloring ingredients may be useful for any of a variety of applications, for example, to increase the appeal of the gummy delivery system to the consumer or to aid in the identification of gummy delivery systems that contain specific active ingredients. In an aspect of the invention, the gummy delivery system is opaque. In a preferred aspect of the invention, the gummy delivery system comprises natural and/or artificial flavoring ingredients to adjust the flavor of the gummy delivery system. Such flavoring ingredients may make the gummy delivery system more favorable to the consumer.

Another aspect of the invention provides a method of preparing any of the inventive edible, discontinuous gummy delivery systems described herein. The inventive gummy delivery systems may be prepared by a multiple step process, wherein separate solutions comprising a sweetener solution, a dispersion medium, and an aqueous gelation solution are combined to create a formulation liquor. The formulation liquor may then be molded, cooled, and dried. The sweetener solution, dispersion medium, and aqueous gelation solution are exemplified as Part A, Part B, and Part C, respectively, in the Examples below.

The inventive method may comprise dispersing collagen peptide particles in an edible liquid dispersion medium to produce a dispersion. The collagen peptide particles may be as described herein with respect to other aspects of the invention. The edible liquid dispersion medium may comprise an edible liquid solution in which collagen peptides are not or are only weakly soluble. In an aspect of the invention, the edible liquid dispersion medium comprises one or more of the edible hygroscopic agents described herein with respect to other aspects of the invention. In an aspect of the invention, the edible liquid dispersion medium comprises one or more of glycerin, sorbitol, and water. In another aspect of the invention, the edible liquid dispersion medium comprises anhydrous glycerin or anhydrous sorbitol. In a preferred aspect of the invention, the edible liquid dispersion medium is anhydrous glycerin. In another preferred aspect of the invention, the edible liquid dispersion medium is a glycerin solution that contains some water, but not enough to permit significant dissolution of the collagen peptides. In another aspect of the invention, the edible liquid dispersion medium comprises anhydrous sorbitol. In another aspect of the invention, the edible liquid dispersion medium comprises a sorbitol solution that contains some water, but not enough to permit significant dissolution of the collagen peptides. In another aspect of the invention, the edible liquid dispersion medium comprises a mixture of glycerin, sorbitol, and/or water.

In an aspect of the invention, the method further comprises heating the dispersion medium before dispersing the collagen peptide particles in the dispersion medium. In one preferred aspect of the invention, wherein the active ingredients are collagen peptides, the method comprises heating the dispersion medium to a temperature of 60 to 75° C. before dispersing the collagen peptide particles in the dispersion medium. In another aspect, the method comprises heating the dispersion medium to a temperature of 50 to 60° C. before dispersing the collagen peptide particles in the dispersion medium. Heating the dispersion medium in excess of 75° C. may increase the occurrence of clumping when the collagen peptides are added to the dispersion medium. At temperatures less than 50° C., the collagen peptide-loaded dispersion medium may become paste-like and difficult to process.

In one preferred aspect of the invention, the method comprises dispersing dried collagen peptides in the heated dispersion medium and mixing for less than 10 minutes. In another aspect of the invention, the method comprises dispersing dried collagen peptides in the heated dispersion medium mixing for a duration of 10 to 30 minutes.

In an aspect of the invention, the method further comprises dry blending one or more nutritional supplement(s), nutraceutical(s), and/or pharmaceutical active ingredient(s) with the collagen peptide particles before dispersing the collagen peptide particles (and the one or more nutritional supplement(s), nutraceutical(s), and/or pharmaceutical active ingredient(s)) in the dispersion medium. The one or more nutritional supplements, nutraceuticals, and/or pharmaceutical active ingredients may be as described herein with respect to other aspects of the invention.

The inventive method further comprises combining the dispersion with a sweetener solution and an aqueous gelation solution to produce a formulation liquor.

The sweetener solution may comprise one or more sweeteners. The sweeteners may be as described herein with respect to other aspects of the invention. In an aspect of the invention, the sweetener solution comprises one or more of maltitol syrup, xylitol, sugar, and corn syrup. The method may comprise preparing the sweetener solution by combining one or more sweeteners with water and boiling the sweetener solution at a temperature from 115 to 125° C. until the Brix exceeds 85%. That mixture is then cooled to approximately 100-105° C. For example, the sweetener solution could be made by adding dried sweetener (e.g., dried sugar) to water or starting with a sweetener syrup (e.g., sugar syrup).

The aqueous gelation solution may comprise one or more edible gelling agents. The edible gelling agents may be as described herein with respect to other aspects of the invention. In a preferred aspect of the invention, the gelling agent is gelatin. Although gelatin is exemplified as the gelling agent (Part C) in the Examples below, it is to be understood that other gelling hydrocolloids can be used as a replacement for the gelatin in the aqueous gelation solution. These other hydrocolloids may include, but are not limited to, starches, pectin, agar, locust bean gum, gellan gum, gum arabic, carrageenan, alginate, modified cellulose and xanthan gum. Only minor modifications, well within the knowledge of one of ordinary skill in the art, would be needed to prepare the inventive gummy delivery systems using these other hydrocolloids.

In an aspect of the invention, the method further comprises dissolving one or more edible gelling agents in water to form the aqueous gelation solution. In one preferred aspect of the invention, the amount of water needed to dissolve the edible gelling agent and prepare the aqueous gelation solution is less than 15% by total solution weight.

The aqueous gelation solution may be prepared by fully dissolving a dried gelling agent (e.g., gelatin) into water at a temperature of 40 to 80° C., with the range of 55 to 65° C. being preferred as higher temperatures may degrade the gelling agent and lower temperatures may have a higher and less desirable viscosity. The gelling agent can comprise from 2% to 15% of the total formulation liquor weight, preferably from 5% to 10% of the total formulation liquor weight.

In an aspect of the invention, the method further comprises heating one or both of the sweetener solution and the aqueous gelation solution prior to combining with the dispersion.

The method may further comprise adding one or more of flavoring ingredient(s), coloring ingredient(s), and pH adjusting ingredient(s) to the formulation liquor. Ingredients useful for imparting a desired color and taste can be added to the sweetener solution through a tailoring cocktail. These can be added to the sweetener solution after it has been cooled. It will also be evident to those skilled in the art that such color and taste imparting ingredients could also be added at other points in the inventive method, such as directly to the aqueous gelation solution or after the aqueous gelation solution, sweetener solution, and dispersion have been combined.

In one preferred aspect of the invention, the sweetener solution and aqueous gelation solution are first combined together and then are mixed with the dispersion. In another preferred aspect of the invention, the dispersion and aqueous gelation solution are first combined together and then are mixed with the sweetener solution. In another preferred aspect of the invention, the sweetener solution and dispersion are first combined together and then are mixed with the aqueous gelation solution. In another aspect of the invention, the dispersion, sweetener solution and aqueous gelation solution are mixed simultaneously.

The inventive method further comprises molding and drying the formulation liquor into a chewable shape, thereby producing the edible, discontinuous gummy delivery system. In an aspect of the invention, the gummy delivery system produced by the method is opaque.

In an aspect of the invention, the total amount of water present in the formulation liquor prior to molding and drying is less than 25% when the aqueous gelation solution comprises gelatin and less than 40% when the aqueous gelation solution comprises starch. In an aspect of the invention, the total amount of water present in the formulation liquor prior to molding and drying is less than 20%, less than 15%, less than 10%, less than 5%, or a range defined by any two of the foregoing values, when the aqueous gelation solution comprises gelatin. In an aspect of the invention, the total amount of water present in the formulation liquor prior to molding and drying is less than 40%, less than 35%, less than 30%, less than 25%, less than 20%, less than 15%, less than 10%, or a range defined by any two of the foregoing values, when the aqueous gelation solution comprises starch.

It may be advantageous for the formulation liquor used to prepare the inventive gummy delivery system to comprise less than 15% water. For example, it may be advantageous for the formulation liquor used to prepare the inventive gummy delivery system to comprise water in an amount less than 15%, less than 14%, less than 13%, less than 12%, less than 11%, less than 10%, less than 9%, less than 8%, less than 7%, less than 6%, less than 5%, or a range defined by any two of the foregoing values. Conventional gummy formulations that contain high loads of collagen peptides typically require approximately 25% or more direct water addition to prepare a fully dissolved and acceptable homogeneous formulation liquor capable of properly molding and drying.

In a preferred aspect of the invention, the formulation liquor used to prepare the inventive gummy delivery systems comprise collagen peptides at a concentration of 20% to 40%. For a 7.5 gram serving size (approximately 3 gummy units of 2500 mg) this concentration corresponds to a 1500-3000 mg dose of collagen peptides. For example, it may be advantageous for the formulation liquor used to prepare the inventive gummy delivery systems to comprise collagen peptides at a concentration of 20%, 25%, 30%, 35%, 40%, or a range defined by any two of the foregoing values. In a preferred aspect of the invention, the formulation liquor used to prepare the inventive gummy delivery system comprises collagen peptides at a concentration of 1% to 40% and additional nutritional supplements are also present in the formulation liquor.

In an aspect of the invention, molding and drying the formulation liquor into the chewable shape further comprises cooling the formulation liquor.

In an aspect of the invention, the method comprises molding the formulation liquor into a chewable shape using a starch-based, silicon, or plastic molding.

In a preferred aspect of the invention, the formulation liquor is produced by running the dispersion, sweetener solution and aqueous gelation solution in separate feed streams, combining them in a flow-through mixing chamber and then depositing the formulation liquor into the desired mold. The formulation liquor, being in a metastable state, may not be stable at the temperature of the separate dispersion, sweetener solution and aqueous gelation solution. Long hold times may dissolve the collagen peptides and may, undesirably, reduce the presence of the desired microstructure in the final gummy delivery system product. Mold sizes typically range in size from 1-5 grams and have a surface to volume ratio sufficient for the gummy delivery system to cool at an acceptable speed. Once cooled to ambient temperatures, the desired discontinuous microstructure may be stable. In another aspect of the invention, the method comprises preparing the formulation liquor in a single mixing tank and depositing it into the desired mold, such that the collagen peptides are limited in their ability to dissolve prior to molding and drying.

By initially dispersing the collagen peptides in an edible liquid dispersion medium as described herein, in which the collagen peptides are not or are only weakly soluble, less or no additional water is required in the formulation liquor. This may provide an advantage in that when the dispersion is combined with the aqueous gelation solution to form the formulation liquor, the gelatin has already been fully dissolved using a standard amount of water. Gelation during cooling locks the system into a stable state. For the delivery of collagen peptides in a gummy delivery system, the amount of water required is significantly reduced. This may provide for easier drying, lower water activity, and a more stable product during prolonged storage.

The following examples further illustrate the invention but, of course, should not be construed as in any way limiting its scope.

### Example 1

This example demonstrates the preparation and characteristics of a sugar-free, discontinuous gummy delivery system for collagen peptides according to an aspect of the invention using a glycerin dispersion medium.

Consumable gummy delivery systems comprising a discontinuous structure and high loads of collagen peptides were prepared as follows. The specific weight concentrations used for four different formulations (Formulations I-IV) of the inventive gummy delivery system are listed in Table A below. A sweetener mixture of maltitol syrup and xylitol was prepared and boiled at 115-125° C. until the Brix reached 87-88%. This mixture was then cooled to 100° C. (Part A). Separately, collagen peptide (VERISOL® collagen peptides (Gelita AG, Eberbach, Germany) supplied as a dry power) was mixed into glycerin at 60° C. to produce a dispersion (Part B). In this example, gelation solutions with a gelatin concentration ranging from 33 to 34% were prepared at a temperature of from 60 to 70° C. (Part C). The gelatin utilized was Type A with a Bloom value of approximately 250 grams. The collagen peptide glycerin dispersion (Part B) was combined with the maltitol syrup/xylitol mixture (Part A), followed by the addition of the gelatin solution (Part C) along with the components listed in Table A as belonging to Part D. These were mixed well. The function of Part D is for pH control and color/flavor expression. As such, Part D included citric acid (50% w/w), lactic acid (85%), colors and flavors. The complete formulation liquor was then deposited into starch molding and dried at room temperature.

The initial formulation liquor concentration of the collagen peptides ranged from 22.1 to 30.5% and was calculated by dividing the mass of collagen peptide by the total weight of initial ingredients. Following molding and drying, the percent weight composition of collagen peptides was further increased following the net removal of moisture.

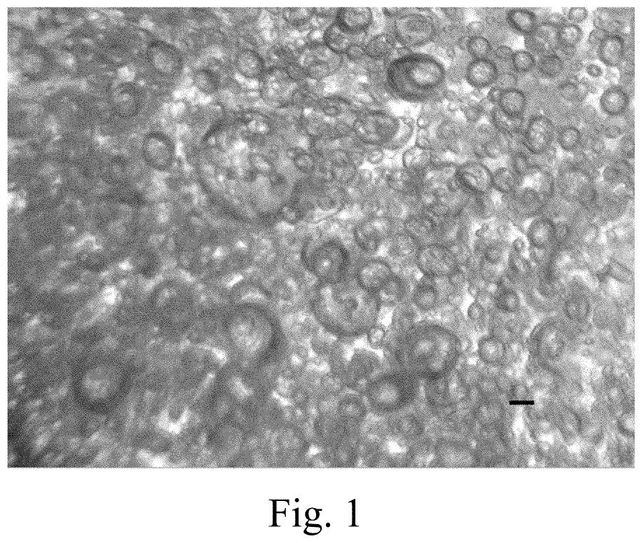

The gummy delivery systems produced were elastic and opaque. FIG. 1 shows a cross section of the prepared delivery system viewed from above and under magnification. The cross-sectional slices were dyed with methyl orange for contrast. The discontinuous microstructure was readily apparent with the dispersed undissolved or partially dissolved collagen peptide particles being clearly visible.

Similar formulations of various active ingredient concentrations, flavors and coloring can also be made by following the specifications provided in Table A. Distinctive color and flavors were incorporated into the formulation. The Control II formulation produced a gummy that was transparent such that a printed grid on a paper placed underneath the gummy was visible through the gummy. In contrast, the printed grid was not visible through the gummy delivery system of inventive formulations I-IV. The components contained in Part D could have been alternatively incorporated into the liquor formulation prior to deposition in a wide variety of alternative methods or processes without loss of function or utility.

TABLE A
Formulations of sugar-free liquors incorporating
various concentrations of collagen peptides.

| | | Control Formulations | | Inventive Formulations | | | |
|---|---|---|---|---|---|---|---|
| Components | Part | I | II | I | II | III | IV |
| 42DE corn syrup | A | 39.87% | — | — | — | — | — |
| Sugar | A | 30.10% | — | — | — | — | — |
| Sorbitol | A | 6.38% | — | — | — | — | — |
| Water | A | 4.49% | — | — | — | — | — |
| Maltitol syrup | A | — | 25.35% | 9.71% | 7.62% | 5.19% | 3.33% |
| Xylitol | A | — | 4.56% | 11.47% | 9.10% | 6.24% | 4.00% |
| Collagen Peptides | B | — | — | 22.06% | 24.77% | 27.3% | 30.49% |
| (VERISOL ®) | | | | | | | |
| Glycerin | B | — | — | 30.88% | 33.35% | 36.21% | 38.11% |
| Water | C | 10.96% | 29.42% | 14.11% | 13.63% | 13.34% | 12.82% |
| Collagen Peptides | C | — | 29.42% | — | — | — | — |
| (VERISOL ®) | | | | | | | |
| Gelatin | C | 5.98% | 5.88% | 7.06% | 6.81% | 6.67% | 6.53% |
| Citric acid (50% w/w) | D | 1.90% | 3.43% | 2.65% | 2.65% | 2.65% | 2.65% |
| Lactic acid (85%) | D | — | 1.47% | 1.50% | 1.50% | 1.50% | 1.50% |
| Color | D | 0.02% | 0.02% | 0.19% | 0.19% | 0.19% | 0.19% |
| Flavor | D | 0.30% | 0.44% | 0.38% | 0.38% | 0.38% | 0.38% |

A conventional control formulation (Control I) was prepared and displayed for comparison. Control I was a conventional, sugar-containing gummy confectionery without collagen peptides, and its formulation is provided in Table A. For Control I, Part A and Part C were prepared separately. There was no Part B. Parts A, C, and D were then combined to produce a well-mixed formulation liquor. The complete formulation liquor was then deposited into starch molding and dried at room temperature in the same manner as described in this Example for inventive Formulations I-IV.

Control II was a sugar-free, collagen peptide-containing gummy delivery system in which the collagen peptides were fully dissolved into the aqueous gelatin solution to prepare a continuous phase. Control II was prepared by dissolving collagen peptide (VERISOL®) into the gelatin precursor solution at 60° C. (Part C). This solution was then combined with a maltitol syrup and xylitol mixture (Part A) as previously described in this Example, deposited into starch molding and allowed to dry at room temperature. The Control II formulation is also provided in Table A. The formula concentration of collagen peptides in Control II was 29.4% and was similar the collagen peptide load in inventive formulations I-IV. To achieve a settable liquor formulation, the amount of water required to dissolve both the gelatin and collagen peptides was significantly higher for Control II relative to inventive Formulations I-IV. The amount of water needed for the Control II formulation was 29.4%, compared to only 12.9-14.0% for inventive Formulations I-IV. The gummy delivery system produced with the Control II formulation was transparent and the discontinuous microstructure was not formed.

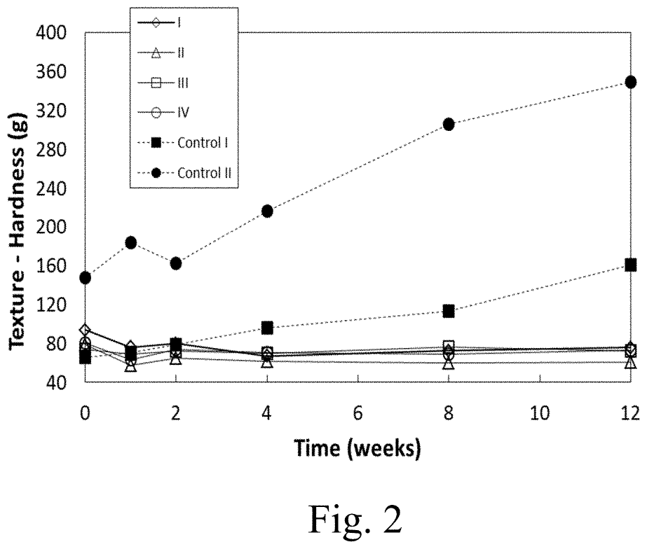

The hardening of consumable gummy delivery systems is a limitation of conventional gummy formulations, as many consumers desire a product that exhibits a soft texture and remains soft over the shelf life of the product. A soft and easily chewable delivery system is also desirable for consumers who have difficulty chewing or swallowing harder objects. The texture of the gummy delivery systems of inventive Formulations I-IV remained more constant over a longer storage period as compared to conventional gummy formulations. FIG. 2 shows the change in texture (hardness) of inventive Formulations I-IV over a 12 week period. During this time, samples were stored in a loosely sealed plastic bag at room temperature.

The texture of the Control II gummy delivery system (continuous, collagen peptide-containing gummy), however, was distinctly harder than the Control I gummy delivery system (conventional sugar-containing gummy that customers prefer). The hardening of the Control II gummy delivery system continued to significantly worsen over time and would be a limitation for consumers accustomed to textures similar to that of the Control I gummy delivery system. The hardness values measured for inventive Formulations I-IV remained significantly softer and were stable during storage.

A gummy delivery system according to an aspect of the present invention, Formulation IV (without colorants), was also prepared as described above and was stored in a loosely sealed plastic bag at room temperature for five years. The texture remained largely unchanged during this exceptionally long storage. The hardness after five years was measured to be 103 g. By contrast, the Control I gummy delivery system exceeded a hardness of 300 g after six months. The Control II gummy delivery system, which contained 29.4% collagen peptides, exceeded a hardness of 457 g after only four months in similar storage conditions. These data demonstrate the exceptional and highly advantageous long-term stability of inventive gummy delivery systems.

The water activity of inventive Formulations I-IV ranged from 0.40 to 0.45 and had an average moisture content of 15.3% after two weeks of storage. After 12 weeks of storage, the water activities of inventive Formulations I-IV were largely unchanged and ranged from 0.44 to 0.46. The average water content of inventive Formulations I-IV was 15.4% after 12 weeks of storage. Control II had a much higher water activity of 0.72, but a similar water content of 15.5% after two weeks of storage, as compared to inventive Formulations I-IV. After 12 weeks of storage, the water activity of Control II had decreased to 0.65 and the moisture had significantly decreased to 10.4%. These results demonstrate that inventive Formulations I-IV significantly limit the ability of water to escape from the gummy delivery system over time.

### Example 2

This example demonstrates the preparation and characteristics of a sugar-containing gummy delivery system for collagen peptides according to an aspect of the invention using a glycerin dispersion medium.

A consumable, gummy delivery system comprising a discontinuous structure and comprising collagen peptides as the active ingredient was prepared as follows. The specific weight concentrations used in this formulation (Formulation V) of the invention are listed in Table B below. A sweetener mixture of sugar and 42DE corn syrup was prepared and boiled at 115-125° C. until the Brix reached 87-88%. This mixture was then cooled to 100° C. (Part A). Separately, collagen peptide (VERISOL® supplied as a dry power) was mixed into glycerin at 60° C. to produce a dispersion (Part B). In this example, the gelation solution was at a 33.3% gelatin concentration and was prepared at a temperature of from 60 to 70° C. (Part C). The gelatin utilized was Type A with a Bloom value of approximately 250 grams. The collagen peptide glycerin dispersion (Part B) was combined with the sugar/42DE corn syrup mixture (Part A), followed by the addition of the gelatin solution (Part C) along with the components listed in Table B as belonging to Part D. These were mixed well. The function of Part D was for pH control and color/flavor expression. As such, Part D included citric acid (50% w/w), lactic acid (85%), colors, and flavors. The complete formulation liquor was then deposited into starch molding and dried at room temperature.

The formulation liquor concentration of the collagen peptide was 21.54% and was calculated by dividing the mass of the collagen peptide by the total weight of initial ingredients.

TABLE B
Formulation of sugar containing
liquor incorporating collagen peptides.

| | | Inventive |
|---|---|---|
| | | Formulation |
| Components | Part | V |
| Sugar | A | 9.48% |
| 42DE corn syrup | A | 11.20% |
| Water | A | 1.72% |
| Collagen Peptides (VERISOL ®) | B | 21.54% |
| Glycerin | B | 30.16% |
| Water | C | 13.79% |
| Gelatin | C | 6.89% |
| Citric acid (50% w/w) | D | 3.27% |
| Lactic acid (85%) | D | 1.72% |
| Color | D | 0.02% |
| Flavor | D | 0.20% |

The gummy delivery system produced was elastic and opaque. Examination under a microscope showed the discontinuous microstructure of the inventive gummy delivery system. The initial hardness was 57.3 g and was only increased to 86.5 g after 12 weeks of storage. The water activity was 0.48 and 0.47 after two and 12 weeks of storage, respectively.

### Example 3

This example demonstrates the preparation and characteristics of a gummy delivery system for collagen peptides according to an aspect of the invention using a sorbitol dispersion medium.

Consumable, gummy delivery systems comprising a discontinuous structure and comprising collagen peptides as the active ingredient were prepared as follows. The specific weight concentrations used in these inventive formulations (Formulation VI and VII) are listed in Table C below. A sweetener mixture of maltitol syrup and xylitol was prepared and boiled at a temperature of from 115 to 125° C. until the Brix reached 87-88%. This mixture was then cooled to 100° C. (Part A). Separately, collagen peptide (VERISOL® supplied as a dry power or FORTIGEL® collagen peptides (Gelita AG, Eberbach, Germany) supplied as a dry powder) was mixed into sorbitol at 60° C. to produce a dispersion (Part B). In this example, the gelation solution was at a 33.7% gelatin concentration and was prepared at a temperature of from 60 to 70° C. (Part C). The gelatin utilized was Type A with a Bloom value of approximately 250 grams. The collagen peptide glycerin dispersion (Part B) was combined with the maltitol syrup/xylitol mixture (Part A), followed by the addition of the gelatin solution (Part C) along with the components listed in Table C as belonging to Part D. These were mixed well. The function of Part D was for pH control and color/flavor expression. As such, Part D included citric acid (50% w/w), lactic acid (85%), colors, and flavors. The complete formulation liquor was then deposited into starch molding and dried at room temperature.

TABLE C
Formulations of sugar-free liquors incorporating
different types of collagen peptides with sorbitol.

| | | Inventive Formulations | |
|---|---|---|---|
| Components | Part | VI | VII |
| Maltitol syrup | A | 3.33% | 3.33% |
| Xylitol | A | 4.00% | 4.00% |
| Collagen Peptides (VERISOL ®) | B | 27.51% | — |
| Collagen Peptides (FORTIGEL ®) | B | — | 27.51% |
| Sorbitol | B | 41.12% | 41.12% |
| Water | C | 12.80% | 12.80% |
| Gelatin | C | 6.52% | 6.52% |
| Citric acid (50% w/w) | D | 2.66% | 2.66% |
| Lactic acid (85%) | D | 1.49% | 1.49% |
| Color | D | 0.19% | 0.19% |
| Flavor | D | 0.38% | 0.38% |

The gummy delivery systems produced were elastic and opaque. Examination under a microscope showed the discontinuous microstructure of the of the inventive gummy delivery system. The initial hardness was found to be 88 g for the inventive Formulation VI and 101.7 g for inventive Formulation VII. The two-week water activity of inventive Formulations VI and VII were 0.51 and 0.60, respectively.

### Example 4

This example demonstrates the preparation and characteristics of a gummy delivery system for collagen peptides with an additional nutritional supplement according to an aspect of the invention using a glycerin dispersion medium.

A consumable, gummy delivery system comprising a discontinuous structure and comprising collagen peptides as the active ingredient and vitamin B2 as a nutritional supplement was prepared as follows.

Sweetener mixtures (Part A) were prepared as denoted in Table D below and were boiled at a temperature of from 115 to 125° C. until the Brix reached 87-88%. This mixture was then cooled to 100° C. Separately, collagen peptides, of varying average MW (and supplied as a dry powder) were mixed into glycerin at 60° C. to produce a dispersion (Part B). In inventive Formulation VIII, the vitamin B2 was dry-blended with the collagen peptides at a concentration of 0.1%. In inventive Formulation IX, powder whey was dry-blended with the collagen peptides at a ratio of 75% collagen peptides to 25% powder whey. In these formulations, the gelation solutions ranged from 26 to 34% gelatin concentration prepared at a temperature of from 60 to 70° C. (Part C). The gelatin utilized was Type A with a Bloom value of approximately 250 grams. The collagen peptide glycerin dispersion (Part B) was combined with the sweetener mixture (Part A), followed by the addition of the gelatin solution (Part C) along with the components listed in Table D as belonging to Part D. These were mixed well. The function of Part D was for pH control and color/flavor expression. The complete formulation liquor was then deposited into starch molding and dried at room temperature.

TABLE D
Formulations of sugar-free liquors incorporating
collagen peptides and additional nutritional supplements.

| | | Inventive Formulations | |
|---|---|---|---|
| Components | Part | VIII | IX |
| Maltitol syrup | A | 3.82% | 3.35% |
| Xylitol | A | 4.40% | 4.02% |
| Collagen Peptides | | | |
| VERISOL ® + Vitamin B2 blend | B | 8.38% | — |
| FORTIGEL ® + Whey | B | — | 30.67% |
| Glycerin | B | 41.91% | 38.33% |
| Water | C | 20.38% | 12.89% |
| Gelatin | C | 7.18% | 6.56% |
| Citric acid (50% w/w) | D | 8.15% | 2.66% |
| Lactic acid (85%) | D | 4.61% | 1.50% |
| Color | D | — | — |
| Flavor | D | 1.17% | — |

The gummy delivery system produced, containing collagen peptides and vitamin B2, was elastic and opaque. Examination under a microscope showed the discontinuous microstructure of the inventive gummy delivery system. The initial water activity was 0.57 units.

The gummy delivery system produced, containing both collagen peptides and whey, was elastic and opaque. Examination under a microscope showed the discontinuous microstructure of the inventive gummy delivery system. The initial water activity was 0.39 units. The average texture of the gummy was also measured to be 97 g.

### Example 5

This example demonstrates the preparation and characteristics of a gummy delivery system for collagen peptides of different molecular weights according to an aspect of the invention using a glycerin dispersion medium.

Consumable, gummy delivery systems comprising a discontinuous structure and comprising collagen peptides were prepared as follows. The specific weight concentrations used for eight different inventive formulations (Formulations SF1-SF4 and SC1-SC4) are listed in Table E and Table F below. Four formulations were sugar-free formulations and are denoted as SF1-SF4. Four formulations contained sugar and are denoted as SC1-SC4.

Sweetener mixtures (Part A) were prepared as denoted in the Tables E-F below and were boiled at a temperature of 115 to 125° C. until the Brix reached 87-88%. This mixture was then cooled to 100° C. Separately, collagen peptides, of varying average molecular weights (and supplied as a dry powder) were mixed into glycerin at 60° C. to produce a dispersion (Part B). In these examples, the gelation solution ranged from 33 to 34% gelatin concentration prepared at a temperature of from 60 to 70° C. (Part C). The gelatin utilized was Type A with a Bloom value of approximately 250 grams. The collagen peptide glycerin dispersion (Part B) was combined with the sweetener mixture (Part A), followed by the addition of the gelatin solution (Part C) along with the components listed in Table E and Table F as belonging to Part D. These were mixed well. The function of Part D was for pH control and color/flavor expression. The complete formulation liquor was then deposited into starch molding and dried at room temperature.

The gummy delivery systems produced were elastic and opaque. The discontinuous microstructure was readily apparent.

The components contained in Part D could have been alternatively incorporated into the liquor formulation prior to deposition in a wide variety of alternative methods or processes without loss of function or utility.

Control formulations of a conventional gummy confectionery and a continuous phase collagen peptide-containing gummy were also prepared for comparison (SF-STD1, SF-STD2, SC-STD1, and SC-STD2). These controls were prepared in the same manner as described in this example with the exception that there was no Part B utilized in production process. In addition, for the control formulations SF-STD2 and SC-STD2, the collagen peptides were fully dissolved along with the gelatin in Part C.

TABLE E
Formulations of sugar-free liquors incorporating
collagen peptides of different molecular weights.

| | | Controls | | Inventive Formulations | | | |
|---|---|---|---|---|---|---|---|
| Components | Part | SF-STD1 | SF-STD2 | SF1 | SF2 | SF3 | SF4 |
| Maltitol syrup | A | 65.71% | 52.00% | 3.34% | 3.34% | 3.34% | 3.34% |
| Xylitol | A | 12.02% | 9.46% | 4.01% | 4.01% | 4.01% | 4.01% |
| Collagen Peptides | | | | | | | |
| MW = 600 Da | B | — | — | 30.55% | — | — | — |
| MW = 2,000 Da | B | — | — | — | 30.55% | — | — |
| MW = 5,000 Da | B | — | — | — | — | 30.55% | — |
| MW = 13,000 Da | B | — | — | — | — | — | 30.55% |
| Glycerin | B | — | — | 38.19% | 38.19% | 38.19% | 38.19% |
| Water | C | 13.12% | 16.84% | 12.84% | 12.84% | 12.84% | 12.84% |
| Collagen Peptides | C | | | | | | |
| MW = 2,000 Da | | — | 12.45% | — | — | — | — |
| Gelatin | C | 7.01% | 5.48% | 6.54% | 6.54% | 6.54% | 6.54% |
| Citric acid (50% w/w) | D | 1.75% | 3.39% | 2.65% | 2.65% | 2.65% | 2.65% |
| Lactic acid (85%) | D | — | — | 1.50% | 1.50% | 1.50% | 1.50% |
| Color | D | — | — | — | — | — | — |
| Flavor | D | 0.38% | 0.38% | 0.38% | 0.38% | 0.38% | 0.38% |

After two weeks, the average hardness was 42.6 g, 37.5 g, 64.1 g and 449.6 g for inventive formulations SF1 through SF4, respectively. The average water activity was 0.442, 0.410, 0.442, and 0.405 for inventive formulations SF1 through SF4, respectively. The sugar free controls, SF-STD1 and SF-STD2, had an average hardness of 129.4 g and 70.9 g, respectively, and an average water activity of 0.672 and 0.704, respectively.

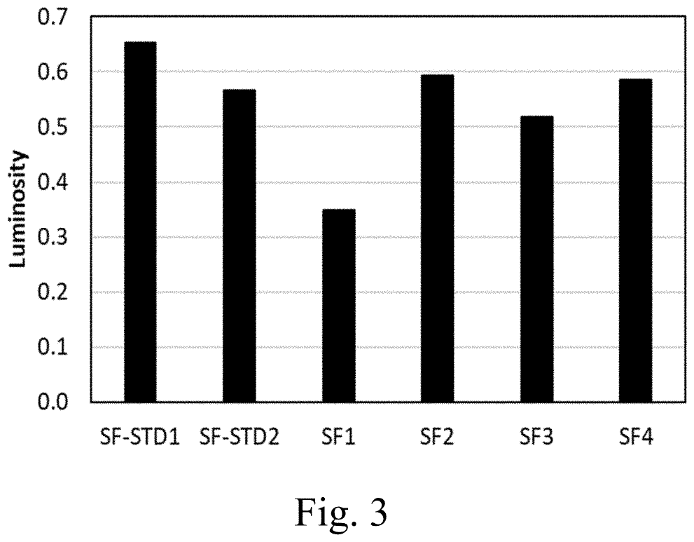

Despite containing significant higher amounts of collagen peptides, the effects of Maillard browning on the inventive sugar-free formulations were similar to or better than conventional collagen peptide-containing gummy formulations. The MW of the collagen peptide affects the rate of Maillard browning with average molecular weights under 1000 Da producing a degree of browning that is undesirable. The luminosity values reported in FIG. 3 were determined by taking photographs of the gummy products, normalizing the red (R), green (G) and blue (B) color values of each pixel, weighting the RGB vector by the relationship 0.299×Red+0.587×Green+0.114×Blue to obtain a luminosity for each pixel, and then averaging the luminosity of each pixel. A luminosity value of 0 denotes black/dark and a luminosity value of 1 denotes white/light. A lower luminosity value corresponds to higher amounts of Maillard browning. A high luminosity value corresponds to lower amounts of Maillard browning. The results are shown in FIG. 3.

TABLE F
Formulations of sugar-containing liquors incorporating
collagen peptides of different molecular weights.

| | | Controls | | Inventive Formulations | | | |
|---|---|---|---|---|---|---|---|
| Components | Part | SC-STD1 | SC-STD2 | SC1 | SC2 | SC3 | SC4 |
| 42DE corn syrup | A | 39.95% | 27.37% | 11.29% | 11.29% | 11.29% | 11.29% |
| Sucrose | A | 30.09% | 24.88% | 9.55% | 9.55% | 9.55% | 9.55% |
| Sorbitol | A | 6.58% | 5.47% | — | — | — | — |
| Water | A | 4.68% | 3.68% | 1.74% | 1.74% | 1.74% | 1.74% |
| Collagen Peptides | | | | | | | |
| MW = 600 Da | B | — | — | 21.70% | — | — | — |
| MW = 2,000 Da | B | — | — | — | 21.70% | — | — |
| MW = 5,000 Da | B | — | — | — | — | 21.70% | — |
| MW = 13,000 Da | B | — | — | — | — | — | 21.70% |
| Glycerin | B | — | — | 30.40% | 30.40% | 30.40% | 30.40% |
| Water | C | 10.96% | 16.22% | 13.90% | 13.90% | 13.90% | 13.90% |
| Collagen Peptides | C | | | | | | |
| MW = 2,000 Da | | — | 13.14% | — | — | — | — |
| Gelatin | C | 5.48% | 5.47% | 6.94% | 6.94% | 6.94% | 6.94% |
| Citric acid (50% w/w) | D | 1.89% | 3.38% | 2.61% | 2.61% | 2.61% | 2.61% |
| Lactic acid (85%) | D | — | — | 1.48% | 1.48% | 1.48% | 1.48% |
| Color | D | — | — | — | — | — | — |
| Flavor | D | 0.38% | 0.38% | 0.38% | 0.38% | 0.38% | 0.38% |

After two weeks, the average moisture of each gummy unit was 21.1%, 17.8%, 18.04%, and 16.8% for inventive formulations SF1 through SF4, respectively. The average hardness was 50.5 g, 38.9 g, 72.8 g, and 295.2 g for inventive formulations SF1 through SF4, respectively. The average water activity was 0.511, 0.488, 0.506, and 0.436 for inventive formulations SF1 through SF4, respectively. An acceptable texture was achieved with a collagen peptide MW upper limit of <13,000 Da. The sugar-free controls, SF-STD1 and SF-STD2, had an average moisture of 12.9% and 24.5%, respectively. The average hardness was 58.7 g and 43.4 g, and the average water activity was 0.693 and 0.739, respectively.

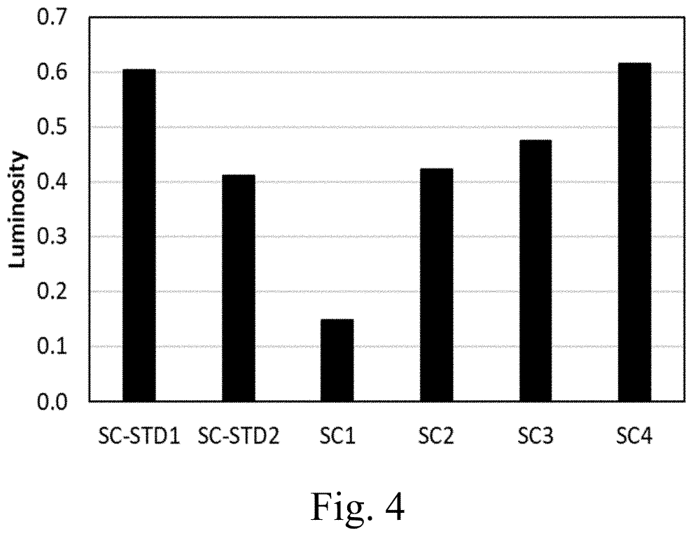

Despite containing significant higher amounts of collagen peptides, the effects of Maillard browning on the inventive sugar-containing formulations were similar to or better than conventional collagen peptide-containing gummies. The MW of the collagen peptide affects the rate of Maillard browning with average molecular weights under 1000 Da producing a degree of browning that is undesirable. The luminosity values reported in FIG. 4 were determined by taking photographs of the gummies, normalizing the red (R), green (G) and blue (B) color values of each pixel, weighting the RGB vector by the relationship 0.299×Red+0.587×Green+0.114×Blue to obtain a luminosity for each pixel, and then averaging the luminosity of each pixel. A luminosity value of 0 denotes black/dark and a luminosity value of 1 denotes white/light. A lower luminosity value corresponds to higher amounts of Maillard browning. A high luminosity value corresponds to lower amounts of Maillard browning. The results are shown in FIG. 4.

The degree of Maillard browning increased with decreasing collagen peptide MW. The initial hardness also increased with increasing MW. This demonstrates that the practical application of the inventive gummy delivery system is most advantageous for collagen peptide components with an average MW of from 1000 to 10,000 Da, which provides a softer texture that is appealing to consumers.

### Example 6

This example demonstrates the preparation and characteristics of a starch-based delivery system for collagen peptides according to an aspect of the invention using a glycerin dispersion medium.

A consumable, gummy delivery system comprising a discontinuous structure and comprising collagen peptides as the active ingredient was prepared as follows. The specific weight concentrations used in this inventive formulation (Formulation X) are listed in Table G below.

A sweetener mixture of maltitol syrup and crystalline maltitol sugar was prepared and boiled at a temperature of from 115 to 125° C. until the Brix reached 87-88%. This mixture was then cooled to 100° C. (Part A). Separately, collagen peptide (VERISOL® supplied as a dry power) was mixed into glycerin at 60° C. to produce a dispersion (Part B). In this example, a starch solution was prepared in a double boiler. The starch was dispersed in the water (Part C) and continuously stirred during cooking until the solution reached boiling. The solution was then held for 10-15 minutes. The sweetener component was then combined with the starch solution and was cooked until the concentration reached 77-78 Brix. This mixture was then combined with the collagen peptide dispersion (Part B) and mixed well. Components needed for flavor, color and pH adjustment (Part D) were added at this point. The complete formulation liquor was then deposited into starch molding and dried in an oven at 50-60° C. for 24-36 hours.

A control sugar-containing starch-based system, without collagen peptides, was similarly prepared. A sweetener mixture of corn syrup 62DE and sucrose was prepared and boiled at 115-125° C. until the Brix reached 87-88%. This mixture was then cooled to 100° C. (Part A). A starch solution was prepared in a double boiler. The starch was dispersed in the water (Part C) and continuously stirred during cooking until the solution reached boiling. The solution was then held for 10-15 minutes. The sweetener component was then combined with the starch solution and was cooked until the concentration reached 77-78 Brix. Components needed for flavor, color and pH adjustment (Part D) were added at this point. The complete formulation liquor was then deposited into starch molding and dried in an oven at 50-60° C. for 24-36 hours.

TABLE G
Formulations of starch-based delivery systems for collagen peptides.

| | | | Inventive |
|---|---|---|---|
| | | Starch | Formulation |
| Components | Part | Control | X |
| Maltitol syrup | A | 28.17% | 18.69% |
| Xylitol | A | 24.65% | 16.36% |
| Corn Syrup 62DE | A | — | — |
| Sucrose | A | — | — |
| Collagen Peptides | B | — | 14.95% |
| Glycerin | B | — | 18.69% |
| Water | C | 38.73% | 25.70% |
| Corn Starch | C | 7.75% | 5.14% |
| Citric acid (50% w/w) | D | 0.70% | 0.47% |
| Lactic acid (85%) | D | — | — |
| Color | D | — | — |
| Flavor | D | — | — |

The formulation liquor concentration of collagen peptide was 14.95% and was calculated by dividing the mass of the collagen peptide by the total weight of initial ingredients.

The control starch-based system had a water activity of 0.59 units and an average hardness of 122 g. The inventive collagen peptide-containing starch-based gummy delivery system had a water activity of 0.38 and an average hardness of 41 g. In this example, the inventive gummy delivery system again produced a high-load collagen peptide gummy delivery system, but without the need to increase the amount of water utilized in the formulation.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An edible, discontinuous gummy delivery system comprising collagen peptide particles with an average particle size from 5 to 500 μm and an average molecular weight from 1,000 to 10,000 Da dispersed in an elastic matrix,
wherein the collagen peptide particles are undissolved or no more than partially dissolved in the elastic matrix, and
wherein the collagen peptide particles are present in an amount from 8 to 45% of a total mass of the delivery system.

2. The gummy delivery system of claim 1, wherein the elastic matrix comprises one or more edible gelling agents, one or more sweeteners, and one or more edible hygroscopic agents.

3. The gummy delivery system of claim 2, wherein the one or more edible gelling agents are selected from the group consisting of gelatin, starch, pectin, agar, locust bean gum, gellan gum, gum arabic, carrageenan, alginate, modified cellulose, and xanthan gum.

4. The gummy delivery system of claim 2, wherein the one or more edible hygroscopic agents is selected from the group consisting of glycerin, sorbitol, anhydrous glycerin, and anhydrous sorbitol.

5. The gummy delivery system of claim 1, further comprising one or more nutritional supplement(s), nutraceutical(s), and/or pharmaceutical active ingredient(s), optionally wherein the one or more nutritional supplements are selected from the group consisting of vitamin A, vitamin B1, vitamin B2, vitamin B6, vitamin B12, vitamin C, vitamin D, vitamin E, vitamin K, biotin, choline, iodine, folic acid, histidine, isoleucine, leucine, lysine, methionine, phenylalanine, threonine, tryptophan, valine, hydroxyproline, calcium, zinc, iron, phosphorous, pea protein, soy, whey and fish oil.

6. The gummy delivery system of claim 1, wherein the gummy delivery system is opaque.

7. The gummy delivery system of claim 1, further comprising one or more ingredients selected from the group consisting of flavoring ingredient(s), coloring ingredient(s), and pH adjusting ingredient(s).

8. A method of preparing the edible, discontinuous gummy delivery system of claim 1, the method comprising:
dispersing collagen peptide particles in an edible liquid dispersion medium to produce a dispersion;
combining the dispersion with a sweetener solution and an aqueous gelation solution to produce a formulation liquor; and
molding and drying the formulation liquor into a chewable shape, thereby producing the edible, discontinuous gummy delivery system.

9. The method of claim 8, where the total amount of water present in the formulation liquor prior to molding and drying is less than 25% when the aqueous gelation solution comprises gelatin and less than 40% when the aqueous gelation solution comprises starch.

10. The method according to claim 8 or 9, further comprising heating the dispersion medium before dispersing the collagen peptide particles in the dispersion medium.

11. The method according to claim 8, further comprising heating one or both of the sweetener solution and the aqueous gelation solution prior to combining with the dispersion.

12. The method according to claim 8, wherein molding and drying the formulation liquor into the chewable shape further comprises cooling the formulation liquor.

13. The method according to claim 8, further comprising dissolving one or more edible gelling agents in water to form the aqueous gelation solution.

14. The method according to claim 8, wherein the aqueous gelation solution comprises one or more edible gelling agents selected from the group consisting of gelatin, starch, pectin, agar, locust bean gum, gellan gum, gum arabic, carrageenan, alginate, modified cellulose, and xanthan gum.

15. The method according to claim 8, further comprising adding one or more of flavoring ingredient(s), coloring ingredient(s), and pH adjusting ingredient(s) to the formulation liquor.

16. The method according to claim 8, wherein the edible liquid dispersion medium comprises one or more edible hygroscopic agents.

17. The method according to claim 8, wherein the edible liquid dispersion medium comprises one or more of glycerin, sorbitol, and water.

18. The method according to claim 8, wherein the edible liquid dispersion medium comprises anhydrous glycerin or anhydrous sorbitol.

19. The method according to claim 8, wherein the sweetener solution comprises one or more of maltitol syrup, xylitol, sugar, and corn syrup.

20. The method according to claim 8, further comprising dry blending one or more nutritional supplements, nutraceuticals, and/or pharmaceutical active ingredients with the collagen peptide particles before dispersing the collagen peptide particles in the dispersion medium.

21. The method according to claim 20, wherein the one or more nutritional supplements are selected from one or more of vitamin A, vitamin B 1, vitamin B2, vitamin B6, vitamin B12, vitamin C, vitamin D, vitamin E, vitamin K, biotin, choline, iodine, folic acid, histidine, isoleucine, leucine, lysine, methionine, phenylalanine, threonine, tryptophan, valine, hydroxyproline, calcium, zinc, iron, phosphorous, pea protein, soy, whey or fish oil.

22. The method according to claim 8, comprising molding the formulation liquor into a chewable shape using a starch-based, silicon, or plastic molding.

23. The method of claim 8, wherein the gummy delivery system produced by the method is opaque.
